(19) United States
(12) Plant Patent Application Publication
(10) Pub. No.: US 2004/0194177 P1
(43) Pub. Date: Sep. 30, 2004

(54) ANGELONIA PLANT NAMED 'CART LAVER'

(76) Inventor: Ralph Tyler Perkins, Gilroy, CA (US)

Correspondence Address: JONDLE & ASSOCIATES P.C., 9085 EAST MINERAL CIRCLE, SUITE 200, CENTENNIAL, CO, 80112, US

(21) Appl. No.: 10/397,982

(22) Filed: Mar. 25, 2003

(51) Int. Cl. A01H 5/00

(52) U.S. Cl. PLT/263

(57) ABSTRACT

An Angelonia cultivar particularly distinguished by lavender color flowers, compact habit and good basal branching.

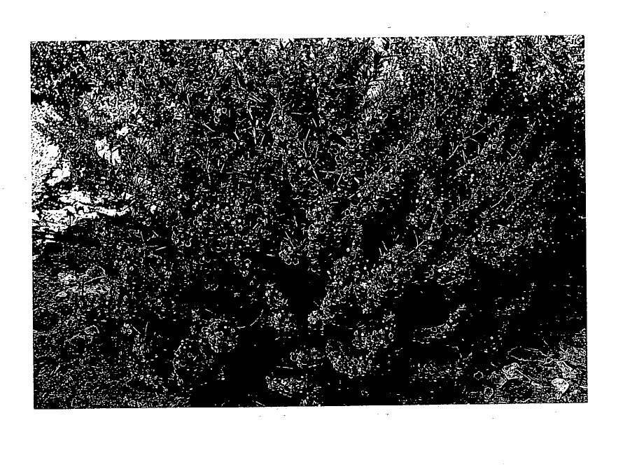

### GENUS AND SPECIES

Angelonia angustifolia

### VARIETY DENOMINATION

‘Cart Laver’

### BACKGROUND OF THE NEW PLANT

The present invention comprises a new and distinct cultivar of Angelonia, botanically known as Angelonia angustifolia and hereinafter referred to by the cultivar name ‘Cart Laver’. The new cultivar is asexually reproduced from vegetative cuttings and tissue culture resulting from the cross of the seed/pod parent ‘AngelMist Purple Stripe’, a purple and white, bicolor commercial line that is unpatented×‘AngelMist Lavender’, a lavender commercial line that is unpatented.

‘Cart Laver’ is a product of a planned breeding program intended to create new angelonia plants with lavender colored flowers, compact habit, good basal branching and moderately vigorous growth.

The new cultivar was created in 2000 in Gilroy, Calif. and has been asexually reproduced repeatedly by vegetative cuttings and tissue culture in Gilroy, Calif., Andijk, The Netherlands, and Guatemala over a two and half-year period. The plant has also been trialed at Gilroy, Calif., Litchfield, Mich. and Andijk, The Netherlands. The present invention has been found to retain its distinctive characteristics through successive propagations; and this novelty is firmly fixed.

### DESCRIPTION OF PHOTOGRAPH

This new Angelonia plant is illustrated by the accompanying photograph which shows blooms, buds, and foliage of the plant in full color, the colors shown being as true as can be reasonably obtained by conventional photographic procedures. The photograph was taken in late summer, of 8-9 month old plants grown in the fields.

The Photograph shows the mature inflorescence.

### DESCRIPTION OF THE NEW CULTIVAR

The following detailed descriptions set forth the distinctive characteristics of ‘Cart Laver’. The data, which defines these characteristics, were collected from asexual reproductions carried out in Gilroy, Calif. The plant history was taken on 4 months old plants grown in one-gallon pots in fall/winter season, in a poly-covered greenhouse under 2-4 supplemental light and color readings were taken in the greenhouse in the winter season under natural light. Plants had been cut back 1-2 times prior to data readings taken. All major branches are cut back about 10-20 cm to keep the plant from becoming unruly. Color references are primarily to the RHS Colour Chart of The Royal Horticultural Society of London (RHS). Texture description details were observed under a dissecting microscope according to The New Royal Horticultural Society Dictionary of Gardening (1992).

### THE PLANT

Classification:

Botanical.—Angelonia angustifolia.

Commercial.—Angelonia.

Form: Upright and erect.

Growth and branching habit: Good vigorous habit, well branched, pinching 1-2 times after transplant enhanced branching.

Height: From soil level to top of blooms: Approximately 40-50 cm.

Width: Approximately 30-45 cm including flowers.

Time to produce a finished flowering plant: 8-10 weeks for a 4-6 inch pot.

Outdoor plant performance: Full sun, free-flowering through the summer, some heat tolerance. Use in mixed container plantings or mass planting in a bed.

Time to initiate roots: Approximately 18-23 days at warm temperatures.

Root description: Fibrous, free branching.

### THE LEAVES

Arrangement: Opposite.

Length: 7.5-8.5 cm.

Width: 1.1-1.2 cm.

Leaf blade shape: Linear.

Leaf margin: Serrate.

Apex shape: Acute.

Base shape: Cuncate.

Mature leaf color: Upper side; RHS 137A (green); Underside, RHS 138A (green) with RHS 144B (yellow-green) mid-vein.

Juvenile leaf color: Upperside between RHS 143A & B; Underside, closest to RHS 144A, but greener.

Texture: Some glandular hairs with a yellow-green hue, physically leaving a sticky residue.

Venation: Pinnate.

Venation color: RHS 144B (yellow-green).

### THE STEM

Length: 40-50 cm from base of plant to tip of branch.

Diameter: 0.3-0.4 cm taken from the lower ⅓ of the stem.

Internodes length: 1.2-2.1 cm.

Branch aspect: 45-60 degree.

Color: RHS 144B (yellow-green).

Texture: Many glandular hairs of various sizes, some with a yellow or orange hue.

Stem anthocyanin: No.

Pedicel color: Upper side, RHS 137A (green) with anthocyanin; Underside, between RHS 137A and B (green).

Pedicel length: 0.8-1.6 cm.

Pedicel diameter: 0.1 cm.

Pedicel texture: Many glandular hairs, some with yellow or orange hue.

Pedicel aspect: 45-60 degrees.

### THE BUD

Shape: Orbicular.

Diameter: 0.5-0.6 cm.

Length: 0.5-0.7 cm.

Color at tight bud: RHS N82B (purple-violet) but a little greyed.

### THE FLOWER

Blooming habit: Continuous throughout the growing season. Good floriferousness.

Floret and Inflorescence type: Single, 2 lipped (5-petaled, fused at base) flowers, solitary or sometimes two in a leaf axis on a terminal raceme.

Flower color, upper side, upper lip/lateral petals: RHS 83C (violet); Mid-petals: RHS 83C (violet) base color with a patch of RHS N155B (white) and RHS 145A (yellow-green), basally.

Flower color, underside: RHS 83D (violet) base color with a spot of RHS N155B (white) basally on the mid-petal.

Corolla color upper portion: RHS N155B (white) base color, heavily overlaid with small spots of RHS N79B (purple).

Corolla color lower portion: RHS 83C (violet) base color with small spots and bars of RHS N79B (purple).

Florets per raceme: 32-40.

Floret diameter: 2.3-2.5 cm.

Petal size, upper lip, 1 petal, and length: 0.5-0.6 cm from the corolla opening; width 0.8-0.9 cm.

Lower lip, lateral petal, and length: 0.5-0.6 cm from the corolla opening; width 0.7-0.8 cm.

Lower lip, mid-petal, length: 1.8-1.9 cm from ovary; width 0.7-0.8 cm.

Petal base: Fused.

Petal margin: Entire.

Petal texture: Papillose and some short glandular hairs.

Sepals: 5, fused at base.

Sepal color: RHS 137A (green).

Sepal length: 0.3-0.4 cm.

Sepal width: 0.2-0.3 cm.

Sepal shape: Ovate.

Sepal apex: Acute.

Sepal anthocyanin: Yes.

Sepal texture: Many glandular hairs with yellow hue.

Lastingness of individual blooms: 6-9 days.

Fragrance: None.

### THE REPRODUCTIVE ORGANS

Stamens: 4, in pairs.

Filament color: RHS N155B (white).

Pollen color: RHS3 N187B (greyed-purple).

Pollen amount: moderate.

Pistil: One.

Stigma color: RHS 145D (yellow-green).

Style color: RHS 145D (yellow-green).

Fruit seed set: Not observed.

### DISEASE AND INSECT RESISTANCE

Not observed.

### COMPARISON WITH KNOWN CULTIVARS

‘Cart Laver’ differs from the female parent, ‘AngelMist Purple Stripe’, in the following ways: ‘Cart Laver’ has a fuller and freer branching habit, freer flowering and is a lavender colored flower and ‘AngelMist Purple Stripe has a white with a purple stripe flower.

‘Cart Laver’ differs from the male parent, ‘AngleMist Lavender’ in the following ways: ‘Cart Laver’ is more upright, has fuller and freer branching habit, has a larger flower than ‘AngelMist Lavender’.

I claim:

1. A new and distinct Angelonia plant as shown and described herein.
